(19) United States
(12) Patent Application Publication
(10) Pub. No.: US 2018/0279905 A1
(43) Pub. Date: Oct. 4, 2018

(54) MAGNETIC RESONANCE IMAGING

(71) Applicant: Shanghai Neusoft Medical Technology Co., Ltd., Shanghai (CN)

(72) Inventor: Cao CHEN, Shanghai (CN)

(21) Appl. No.: 15/938,868

(22) Filed: Mar. 28, 2018

(30) Foreign Application Priority Data
Mar. 28, 2017 (CN) 201710193751.3

(51) Int. Cl. A61B 5/055; G01R 33/565; G01R 33/56; G01R 33/48; G01R 33/483

(52) U.S. Cl. CPC A61B 5/055; G01R 33/56527; G01R 33/4838; G01R 33/4828; G01R 33/5607

(57) ABSTRACT

Methods and devices for magnetic resonance imaging are provided. In one aspect, a method of magnetic resonance imaging includes: for each scanning step in a scanning sequence for a subject, determining a longitudinal magnetization intensity of a characteristic tissue of the subject according to a current running condition of the scanning sequence, obtaining an inversion time corresponding to a characteristic tissue suppressing pulse to be generated according to the determined longitudinal magnetization intensity, generating the characteristic tissue suppression pulse, generating an imaging pulse sequence when the inversion time elapses after generating the characteristic tissue suppression pulse, and receiving an echo signal from the subject, the echo signal corresponding to the generated imaging pulse sequence. The method can also include reconstructing an image of the subject according to the received echo signals when the scanning sequence finishes the scanning steps.

### CROSS REFERENCE TO RELATED APPLICATIONS

This application claims priority to Chinese Patent Application No. 201710193751.3 entitled “Method and Device for Magnetic Resonance Imaging” filed on Mar. 28, 2017, the entire content of which is incorporated herein by reference.

### TECHNICAL FIELD

The present disclosure relates to magnetic resonance imaging.

### BACKGROUND

Magnetic Resonance Imaging (MRI), as a multi-parameter and multi-contrast imaging technology, is an imaging method in modern medical imaging. A plurality of characteristics (such as longitudinal relaxation time T₁, transverse relaxation time T₂ and proton density of a tissue) can be reflected through the MRI. Thus, information for disease detection and disease diagnosis can be provided through the MRI. For the MRI, hydrogen protons in a subject are excited through radio-frequency excitation by using magnetic resonance, position coding is performed by using a gradient field, an electromagnetic signal carrying position information is received via a receiving coil, and image information is reconstructed by Fourier transform.

NEUSOFT MEDICAL SYSTEMS CO., LTD. (NMS), founded in 1998 with its world headquarters in China, is a leading supplier of medical equipment, medical IT solutions, and healthcare services. NMS supplies medical equipment with a wide portfolio, including CT, Magnetic Resonance Imaging (MRI), digital X-ray machine, ultrasound, Positron Emission Tomography (PET), Linear Accelerator (LINAC), and biochemistry analyser. Currently, NMS' products are exported to over 60 countries and regions around the globe, serving more than 5,000 renowned customers. NMS's latest successful developments, such as 128 Multi-Slice CT Scanner System, Superconducting MRI, LINAC, and PET products, have led China to become a global high-end medical equipment producer. As an integrated supplier with extensive experience in large medical equipment, NMS has been committed to the study of avoiding secondary potential harm caused by excessive X-ray irradiation to the subject during the CT scanning process.

### BRIEF DESCRIPTION OF DRAWINGS

FIG. 1 is a flowchart illustrating a process of a method of magnetic resonance imaging according to an example of the present disclosure.

FIG. 2 is a flowchart illustrating a process of determining longitudinal magnetization intensity and inversion time according to an example of the present disclosure.

FIG. 3 is a schematic diagram illustrating a radio-frequency pulse sequence relative to time and a relaxation curve of the longitudinal magnetization intensity of a characteristic tissue according to an example of the present disclosure.

FIG. 4 is a diagram illustrating a fat signal suppressing effect by using fixed inversion time in a fat signal suppressing experiment according to an example of the present disclosure.

FIG. 5 a diagram illustrating a fat signal suppressing effect by changing inversion time in a fat signal suppressing experiment according to an example of the present disclosure.

FIG. 6 is a schematic diagram illustrating a structure of a device for magnetic resonance imaging according to an example of the present disclosure.

### SUMMARY

The present disclosure provides methods and devices for magnetic resonance imaging in a way that image scanning efficiency can be improved.

One aspect of the present disclosure features a method of magnetic resonance imaging, including: for each scanning step in a scanning sequence for a subject, performing operations including: determining a longitudinal magnetization intensity of a characteristic tissue of the subject according to a current running condition of the scanning sequence; obtaining an inversion time corresponding to a characteristic tissue suppressing pulse to be generated according to the determined longitudinal magnetization intensity; generating the characteristic tissue suppression pulse; generating an imaging pulse sequence when the inversion time elapses after generating the characteristic tissue suppression pulse; and receiving an echo signal from the subject, the echo signal corresponding to the generated imaging pulse sequence. The method also includes reconstructing an image of the subject according to the received echo signals when the scanning sequence finishes the scanning steps. The scanning step can be a layer when the scanning sequence is a two-dimensional scanning sequence or a slice selection direction code when the scanning sequence is a three-dimensional scanning sequence.

In some implementations, determining the longitudinal magnetization intensity of the characteristic tissue includes: in response to determining that the longitudinal magnetization of the characteristic tissue does not reach a stable state, estimating the longitudinal magnetization intensity of the characteristic tissue according to the current running condition of the scanning sequence and an expected longitudinal magnetization intensity of the characteristic tissue in the stable state. The expected longitudinal magnetization intensity of the characteristic tissue in the stable state can be determined according to a repetition time of the characteristic tissue suppressing pulse and a longitudinal relaxation time of the characteristic tissue.

In some implementations, determining the longitudinal magnetization intensity of the characteristic tissue includes: in response to determining that the longitudinal magnetization of the characteristic tissue reaches a stable state, determining the longitudinal magnetization intensity of the characteristic tissue based on a repetition time of the characteristic tissue suppressing pulse and a longitudinal relaxation time of the characteristic tissue.

In some implementations, determining the longitudinal magnetization intensity of the characteristic tissue includes: in response to determining that an event of disturbing a stable state of the longitudinal magnetization occurs, determining the longitudinal magnetization intensity of the characteristic tissue according to the event of disturbing the stable state of the longitudinal magnetization.

In some cases, determining the longitudinal magnetization intensity of the characteristic tissue according to the event of disturbing the stable state of the longitudinal magnetization can include: determining the current running condition of the scanning sequence; determining whether an idle time of the scanning sequence is greater than a predetermined threshold; in response to determining that the idle time is no greater than the predetermined threshold, determining the longitudinal magnetization intensity to be an experiential value; and in response to determining that the idle time is greater than the predetermined threshold, calculating the longitudinal magnetization intensity in real time according to the determined current running condition of the scanning sequence.

In some implementations, obtaining the inversion time corresponding to the characteristic tissue suppressing pulse to be generated according to the determined longitudinal magnetization intensity includes: determining the inversion time according to the determined longitudinal magnetization intensity and an expected residual longitudinal magnetization intensity of the characteristic tissue after generating the characteristic tissue suppressing pulse, such that the longitudinal magnetization intensity of the characteristic tissue is substantially equal to the expected longitudinal magnetization intensity of the characteristic tissue when the inversion time elapses after generating the characteristic tissue suppressing pulse.

In some implementations, obtaining the inversion time corresponding to the characteristic tissue suppressing pulse to be generated according to the determined longitudinal magnetization intensity includes: in response to determining that the longitudinal magnetization of the characteristic tissue does not reaches a stable state, determining the inversion time corresponding to the characteristic tissue suppressing pulse according to the determined longitudinal magnetization intensity; and in response to determining that the longitudinal magnetization of the characteristic tissue reaches the stable state, setting the inversion time corresponding to the characteristic tissue suppressing pulse to be a fixed value. Determining the inversion time corresponding to the characteristic tissue suppressing pulse according to the determined longitudinal magnetization intensity can include: determining the inversion time based on the determined longitudinal magnetization intensity and an expected residual longitudinal magnetization intensity of the characteristic tissue.

Obtaining the inversion time corresponding to the characteristic tissue suppressing pulse to be generated according to the determined longitudinal magnetization intensity can include: adjusting a value of the inversion time for a plurality of times; in response to determining that a number of the plurality of times is more than a predetermined threshold, determining whether the longitudinal magnetization reaches a stable state; and in response to determining that the longitudinal magnetization reaches the stable state, determining the inversion time to be a fixed value.

Another aspect of the present disclosure features a device for magnetic resonance imaging, including: a pulse generator configured to generate a characteristic tissue suppressing pulse and generate an imaging sequence when an inversion time elapses after the characteristic tissue suppression pulse; a processor and a non-transitory machine-readable storage medium storing machine executable instructions which are executable by the processor to: for each scanning step of a scanning sequence for a subject, determine a longitudinal magnetization intensity of a characteristic tissue of the subject according to a current running condition of the scanning sequence and obtain the inversion time corresponding to a characteristic tissue suppressing pulse to be generated according to the determined longitudinal magnetization intensity; a receiving coil configured to receive an echo signal from the subject; and an image reconstructing module configured to reconstruct an image according to the received echo signals when the scanning sequence finishes the scanning steps.

In some implementations, the processor is caused by the machine-executable instructions to: in response to determining that the longitudinal magnetization of the characteristic tissue does not reach a stable state, estimate the longitudinal magnetization intensity of the characteristic tissue according to the current running condition of the scanning sequence and an expected longitudinal magnetization intensity of the characteristic tissue in the stable state. The expected longitudinal magnetization intensity of the characteristic tissue in the stable state can be determined according to a repetition time of the characteristic tissue suppressing pulse and a longitudinal relaxation time of the characteristic tissue.

In some implementations, the processor is caused by the machine-executable instructions to: in response to determining that the longitudinal magnetization of the characteristic tissue reaches a stable state, determine the longitudinal magnetization intensity of the characteristic tissue based on a repetition time of the characteristic tissue suppressing pulse and a longitudinal relaxation time of the characteristic tissue.

In some implementations, the processor is caused by the machine-executable instructions to: in response to determining that an event of disturbing a stable state of the longitudinal magnetization occurs, determine the longitudinal magnetization intensity of the characteristic tissue according to the event of disturbing the stable state of the longitudinal magnetization.

In some implementations, the processor is caused by the machine-executable instructions to: determine the inversion time according to the determined longitudinal magnetization intensity and an expected residual longitudinal magnetization intensity of the characteristic tissue after generating the characteristic tissue suppressing pulse, such that the longitudinal magnetization intensity of the characteristic tissue is substantially equal to the expected longitudinal magnetization intensity of the characteristic tissue when the inversion time elapses after generating the characteristic tissue suppressing pulse.

In some implementations, the processor is caused by the machine-executable instructions to: in response to determining that the longitudinal magnetization of the characteristic tissue does not reaches a stable state, determine the inversion time corresponding to the characteristic tissue suppressing pulse according to the determined longitudinal magnetization intensity; and in response to determining that the longitudinal magnetization of the characteristic tissue reaches the stable state, set the inversion time corresponding to the characteristic tissue suppressing pulse to be a fixed value. The processor can be caused by the machine-executable instructions to: determining the inversion time based on the determined longitudinal magnetization intensity and an expected residual longitudinal magnetization intensity of the characteristic tissue.

In some implementations, the processor is caused by the machine-executable instructions to: adjust a value of the inversion time for a plurality of times; in response to determining that a number of the plurality of times is more than a predetermined threshold, determine whether the longitudinal magnetization reaches a stable state; and in response to determining that the longitudinal magnetization reaches the stable state, determine the inversion time to be a fixed value.

The details of one or more examples of the subject matter described in the present disclosure are set forth in the accompanying drawings and description below. Other features, aspects, and advantages of the subject matter will become apparent from the description, the drawings, and the claims. Features of the present disclosure are illustrated by way of example and not limited in the following figures, in which like numerals indicate like elements.

### DETAILED DESCRIPTION

For MRI, signal intensity of a specific tissue can be excessively high, which has an effect on clinical diagnosis. For example, a fat tissue has high proton density, short longitudinal relaxation time T1 (e.g., from 200 ms to 250 ms under field intensity of 1.5 T), and long transverse relaxation time T2. Thus, the fat tissue has high signal intensity on images with different contrasts. However, the characteristics of the fat tissue can reduce the contrasts among tissues in the images, which may cause an effect on disease detection.

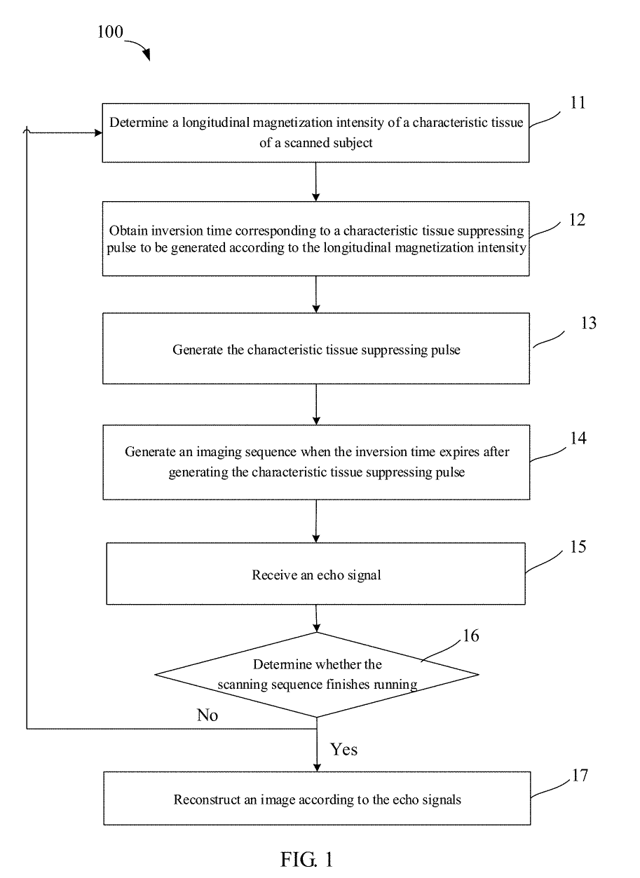

FIG. 1 is a flowchart illustrating a process 100 of a method of a magnetic resonance imaging according to an example of the present disclosure. The method of magnetic resonance imaging can be used to perform imaging on a subject. As shown in FIG. 1, the process 100 includes steps as follows.

At step 11, a longitudinal magnetization intensity of a characteristic tissue of a scanned subject is determined.

The characteristic tissue may be a tissue (e.g., a fat tissue) emitting a high intensity signal which significantly interferes with a signal from another tissue to be imaged. A characteristic tissue suppressing pulse may be a frequency selective saturation pulse (or called as a chemical shift selective RF pulse), which may suppress the characteristic tissue signal through a frequency selective saturation method. In an example, the longitudinal magnetization intensity of the characteristic tissue of the scanned subject is determined according to a current running condition of a scanning sequence.

The longitudinal magnetization intensity of the characteristic tissue before generating a characteristic tissue suppressing pulse may be a longitudinal relaxation value at a time which is closest to and before a time of generating the characteristic tissue suppressing pulse. When the longitudinal magnetization of the characteristic tissue reaches a stable state, the longitudinal magnetization intensity of the characteristic tissue may be determined by formula (1):

$${M}_{z , ss} = \frac{1 - {e}^{\frac{- {TR}_{sup}}{{T}_{1}}}}{1 + {e}^{\frac{- {TR}_{sup}}{{T}_{1}}}} \qquad ( 1 )$$

In formula (1), M_{z,ss} represents a normalized longitudinal magnetization intensity in the stable state, a value of M_{z,ss} is from −1 to 1, TRₛᵤₚ represents repetition time of the characteristic tissue suppressing pulse, and T₁ represents longitudinal relaxation time of the characteristic tissue.

When an event of disturbing the stable state of the longitudinal magnetization (such as a starting phase of an RF (radio frequency) pulse sequence, gating, or RF pulse sequence pausing) occurs, the longitudinal magnetization intensity of the characteristic tissue is not in the stable state. The longitudinal magnetization intensity of the characteristic tissue before generating the characteristic tissue suppression pulse may be determined through an experiential value, or may be calculated in real time. For example, in a starting phase of the RF pulse sequence, an experiential value may be used as the longitudinal magnetization intensity before generating the characteristic tissue suppressing pulse. When the gating is used for suppressing motion artifact, the longitudinal magnetization intensity before generating the characteristic tissue suppressing pulse may be estimated based on gating time and the longitudinal magnetization intensity in the stable state. When the RF pulse sequence pauses, the longitudinal magnetization intensity before generating the characteristic tissue suppressing pulse may be estimated based on pausing time and the longitudinal magnetization intensity in the stable state, etc.

The longitudinal magnetization intensity before generating the characteristic tissue suppressing pulse may be determined through a model. Alternatively, the longitudinal magnetization intensity before generating the characteristic tissue suppression pulse may be estimated or calculated in real time. A method of determining longitudinal magnetization intensity of a characteristic tissue before generating a characteristic tissue suppressing pulse can be selected according to the event of disturbing the stable state of the longitudinal magnetization, a practical application, performance of an applied system.

At step 12, an inversion time corresponding to a characteristic tissue suppressing pulse to be generated is obtained according to the longitudinal magnetization intensity.

The inversion time can be defined as a time period from a time when the characteristic tissue suppressing pulse is generated to a time when the longitudinal magnetization intensity of the characteristic tissue is attenuated to an expected residual longitudinal magnetization intensity of the characteristic tissue. The inversion time may be changed when the longitudinal magnetization intensity is changed.

The inversion time is determined based on the longitudinal magnetization intensity of the characteristic tissue before generating the characteristic tissue suppressing pulse. The longitudinal magnetization intensity before applying the characteristic tissue suppressing pulse to the subject may be changed in a case of disturbing the stable state of the longitudinal magnetization such as the starting phase of the RF pulse sequence, the gating, the RF sequence pausing. For example, the respective longitudinal magnetization intensities which are before sequentially generating the plurality of characteristic tissue suppressing pulses may be different from each other. The respective inversion time corresponding to the characteristic tissue suppressing pulses may be sequentially adjusted according to the longitudinal magnetization intensities before generating the plurality of characteristic tissue suppression pulses. Thus, the respective inversion time corresponding to the characteristic tissue suppressing pulses may be changed when the corresponding longitudinal magnetization intensities before generating the characteristic tissue suppressing pulse are changed. For each characteristic tissue suppressing pulse, the longitudinal magnetization intensity of the characteristic tissue when the corresponding inversion time elapses after generating the characteristic tissue suppressing pulse can be equal to or approximate to the expected residual longitudinal magnetization intensity of the characteristic tissue after generating the characteristic tissue suppressing pulse, e.g., a characteristic tissue suppressing degree. After the longitudinal magnetization of the characteristic tissue is inverted through the characteristic tissue suppressing pulse, a signal of the characteristic tissue may be attenuated to an expected residual characteristic tissue signal through longitudinal relaxation in the inversion time.

In an example, the expected residual longitudinal magnetization intensity of the characteristic tissue is equal to 0, and it is desired the characteristic tissue signal is completely suppressed. In another example, the expected residual longitudinal magnetization intensity of the characteristic tissue is not equal to 0, and it is desired not to completely suppress the characteristic tissue signal. Also after the characteristic tissue is suppressed, it is expected that there is still a left signal from the characteristic issue. The characteristic tissue suppressing degree can be determined based on a requirement of a practical application.

In an example, the longitudinal magnetization intensity M_{z,n} of the characteristic tissue after generating the characteristic tissue suppressing pulse may be expressed through formula (2):

$${M}_{z , n} ( t ) = 1 - ( 1 + {M}_{z , pre , n} ) \cdot {e}^{\frac{- t}{{T}_{1}}} \qquad ( 2 )$$

In formula (2), t represents a time after generating the characteristic tissue suppressing pulse, n represents a n-th characteristic tissue suppressing pulse, M_{z,n}(t) represents the longitudinal magnetization intensity of the characteristic tissue at a time t after generating the n-th characteristic tissue suppressing pulse, and M_{z,pre,n} represents the longitudinal magnetization intensity of the characteristic tissue before generating the n-th characteristic tissue suppressing pulse. M_{z,n} and M_{z,pre,n} may be normalized longitudinal magnetization intensities, a value of which is from −1 to 1.

In an example, the corresponding inversion time is determined according to the longitudinal magnetization intensity of the characteristic tissue before generating the characteristic tissue suppressing pulse and an expected residual longitudinal magnetization intensity of the characteristic tissue after generating the characteristic tissue suppressing pulse. As noted above, the inversion time may be a time period from a time when the characteristic tissue suppressing pulse is generated to a time when the longitudinal magnetization intensity of the characteristic tissue is attenuated to the expected residual longitudinal magnetization intensity of the characteristic tissue. Formula (3) of the inversion time TIₙ corresponding to the n-th characteristic tissue suppressing pulse can be determined based on formula (2):

$${TI}_{n} = {T}_{1} \cdot ln ( \frac{1 + {M}_{z , pre , n}}{1 - {M}_{z , res}} ) \qquad ( 3 )$$

In formula (3), M_{z,res} represents the expected residual longitudinal magnetization intensity of the characteristic tissue, which is a normalized value from −1 to 1. When the expected residual longitudinal magnetization intensity of the characteristic tissue is equal to 0, the inversion time TIₙ can be further expressed as formula (4):

TIₙ=T₁·ln(1+M_{z,pre,n}) (4)

In an example, when the longitudinal magnetization of the characteristic tissue does not reach a stable state, the inversion time corresponding to the characteristic tissue suppressing pulse may be adjusted according to the longitudinal magnetization intensity before generating the characteristic tissue suppressing pulse. When the longitudinal magnetization intensity before generating the characteristic tissue suppression pulse is changed, the inversion time corresponding to the characteristic tissue suppressing pulse is changed. When the longitudinal magnetization of the characteristic tissue reaches the stable state, the inversion time corresponding to the characteristic tissue suppressing pulse may be set with a fixed value. Thus, it can be ensured that the longitudinal magnetization intensity of the characteristic tissue is equal to or approximate to the expected longitudinal magnetization intensity of the characteristic tissue when the inversion time elapses regardless whether the longitudinal magnetization of the characteristic tissue is in the stable state. In an example, it is tracked in real time whether the longitudinal magnetization of the characteristic tissue reaches the stable state. In another example, the number of the characteristic tissue suppressing pulses generated before the longitudinal magnetization of the characteristic tissue reaches the stable state is determined by predicting a longitudinal relaxation condition based on an event condition of disturbing the stable state in the RF pulse sequence, the longitudinal magnetization intensity at a time when the event occurs and the longitudinal relaxation time of the characteristic tissue. Thus, the inversion time corresponding to the characteristic tissue suppressing pulses generated before the longitudinal magnetization of the characteristic tissue reaching the stable state may be adjusted, and the inversion time corresponding to another characteristic tissue suppression pulse is set with a fixed value. For example, a plurality of respective inversion times corresponding to characteristic tissue suppressing pulses generated in the starting phase of the RF pulse sequence (e.g., 3 to 4) are determined, and the inversion time corresponding to the characteristic tissue suppressing pulse generated after the plurality of characteristic tissue suppression pulses is set with the fixed value.

In another example, the inversion time corresponding to each characteristic tissue suppressing pulse is changed according to the corresponding longitudinal magnetization intensity. For example, the inversion time corresponding to each characteristic tissue suppressing pulse is adjusted, such that the longitudinal magnetization intensity of the characteristic tissue can reach the expected residual longitudinal magnetization intensity of the characteristic tissue when the inversion time elapses. When the longitudinal magnetization reaches the stable state, the inversion time may be finely adjusted, such that the longitudinal magnetization intensity can be equal to the expected residual longitudinal magnetization intensity of the characteristic tissue when the inversion time elapses. In an example, the inversion time for each imaging layer is adjusted according to a corresponding longitudinal magnetization intensity before generating the characteristic tissue suppression pulse. Each imaging layer corresponds to a characteristic tissue suppressing pulse for suppressing a characteristic tissue signal corresponding to the imaging layer, which is used for a two-dimensional (2D) imaging sequence. In another example, inversion time for each slice selection direction code is adjusted according to the corresponding longitudinal magnetization intensity before generating the characteristic tissue suppression pulse. Each slice selection direction code corresponds to a characteristic tissue suppression pulse for suppressing a characteristic tissue signal corresponding to the slice selection direction code, which is used for a three-dimensional (3D) imaging sequence.

At step 13, the characteristic tissue suppressing pulse is generated.

The characteristic tissue suppressing pulse may be repeatedly generated based on a repetition time. For example, the characteristic tissue suppression pulses to be applied to a subject in a static magnetic field are sequentially generated based on the repetition time, so as to repeatedly suppress a signal corresponding to the characteristic tissue of the subject. As noted above, the characteristic tissue can be a tissue (e.g., a fat tissue) emitting a high intensity signal which significantly interferes with a signal from another tissue to be imaged.

The characteristic tissue suppressing pulse may be a frequency selective saturation pulse (or called as a chemical shift selective RF pulse), which may suppress the characteristic tissue signal through a frequency selective saturation method. In the frequency selective saturation method, frequency selective inversion recovery is used based on a chemical shift effect of the characteristic tissue and another tissue, such that a characteristic tissue signal is suppressed, and the another tissue signal is not affected. For example, a Larmor frequency of fat protons is slightly lower than a Larmor frequency of water protons, and a frequency difference between the fat protons and the water protons can be from 3.3 to 3.5 ppm. When imaging is performed for water, the fat may be pre-saturated to eliminate the interference for the fat. A 90° RF pulse may be selected for the fat frequency, where a center frequency of the 90° RF pulse aligns to the Larmor frequency of the fat protons, and a bandwidth of the 90° RF pulse is no more than 3.4 ppm. The fat signal may be suppressed as follows. Magnetization intensity of whole-space fat is excited to a transverse plane. The magnetization intensity of the fat in the transverse plane is destructed through a destruction gradient. To perform imaging for the fat, water component may be saturated.

At step 14, an imaging sequence is generated when the inversion time elapses after generating the characteristic tissue suppressing pulse.

The characteristic tissue suppressing pulse is used to invert the longitudinal magnetization of the characteristic tissue. After longitudinal relaxation in the inversion time, the longitudinal magnetization intensity of the characteristic tissue may be equal to or approximate to the expected residual longitudinal magnetization intensity of the characteristic tissue, a characteristic tissue signal is attenuated to be equal to or approximate to the expected residual characteristic tissue signal, and the imaging sequence may be generated. Thus, the characteristic tissue signal can be suppressed.

When the expected residual longitudinal magnetization intensity of the characteristic tissue is equal to 0, the characteristic tissue signal may be attenuated to be 0 after the longitudinal relaxation in the inversion time, and the imaging sequence may be applied. Thus, it can be implemented to completely suppress the characteristic tissue signal. When the expected residual longitudinal magnetization intensity of the characteristic tissue is not equal to zero, the expected residual characteristic tissue signal is not equal to zero. After the longitudinal relaxation in the inversion time, the characteristic tissue signal may be attenuated to the expected residual characteristic tissue signal, and the imaging sequence may be applied when the characteristic tissue signal is attenuated to the expected residual characteristic tissue signal. Thus, it can be implemented to partially suppress the characteristic tissue signal.

The imaging sequence may include an exciting pulse. In an example, the imaging sequence further includes a refocusing pulse. In another example, a gradient field is simultaneously applied to be overlapped on a static magnetic field when the imaging sequence is applied.

At step 15, an echo signal is received.

The echo signal from the subject may be received in a designated time period after the imaging sequence is generated. Since the signal from the characteristic tissue is completely or partially suppressed, the echo signal may not include echo signal from the characteristic tissue, or may include an echo signal with a weaken intensity from the suppressed characteristic tissue. Thus, the echo signal corresponding to one or more tissues to be imaged can be obtained, and the interference from the characteristic tissue signal with the signal of the tissues to be imaged can be avoided or decreased.

At step 16, it is determined whether a scanning sequence for the subject finishes running. The subject includes a plurality of tissues to be imaged. The scanning sequence can include a plurality of scanning steps. As discussed below, when the scanning sequence is a two-dimensional pulse sequence, the scanning step is a layer; when the scanning sequence is a three-dimensional pulse sequence, the scanning step is a slice selection direction code. When the scanning sequence is running, for each scanning step, respective echo signals from the tissues can be received according to the above steps 11-16.

When the scanning sequence does not finish running, e.g., the scanning steps are not completed, the process 100 returns to step 11; otherwise, the process 100 continues to step 17.

At step 17, an image is reconstructed according to the echo signals.

In an example, the image is reconstructed according to the received echo signals when the scanning sequence finishes the scanning steps.

When the subject is scanned layer by layer through a generated two-dimensional RF pulse sequence, a two-dimensional image is reconstructed according to the echo signals. When the subject is scanned slice selection direction code by slice selection orientation code through a generated three-dimensional RF pulse sequence, a three-dimensional image is reconstructed according to the echo signals. Fourier transform may be performed on the received echo signals, and a modular value is obtained to determine a projection intensity signal for image reconstruction. Since the echo signals from the characteristic tissue are suppressed, a high contrast of the tissues in a reconstructed image can be achieved, and a high image quality can further be achieved.

According to examples of the present disclosure, when the longitudinal magnetization of the characteristic tissue does not reach the stable state, the characteristic tissue can be stably suppressed by changing the inversion time in real time. It is not in demand to suppress the characteristic tissue until the longitudinal magnetization of the characteristic tissue reaches the stable state. Further, it is not in demand to discard the echo signal and imaging data obtained when the longitudinal magnetization does not reach the stable state. Thus, imaging scanning time can be fully used to improve imaging scanning efficiency. Further, it is not in demand to apply an additional preparatory pulse for fast reaching the stable state when the stable state is not reached, thus, a specific Absorption Rate (SAR) of the tissue cannot be increased.

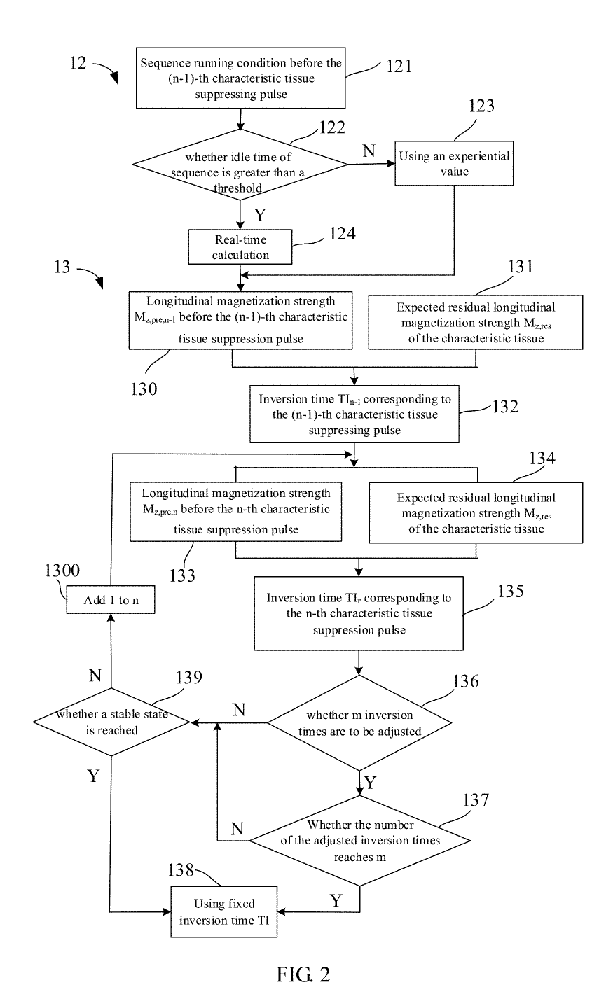

FIG. 2 illustrates a process in steps 11 and 12 of the method of magnetic resonance imaging in FIG. 1 according to an example of the present disclosure. The step 11 in FIG. 2 includes sub-steps 121-124, and the step 12 includes sub-steps 130-139 and 1300, which are described below in detail in conjunction with the drawings.

At sub-step 121, when an event of disturbing a stable state of longitudinal magnetization occurs for an RF pulse sequence, a current running condition of the RF pulse sequence is determined. The current running condition of the RF pulse sequence may include a last longitudinal relaxation value in the repetition time of the current characteristic tissue suppressing pulse. It is assumed that the event of disturbing the stable state of the longitudinal magnetization occurs before a (n−1)-th characteristic tissue suppressing pulse, where n is a positive integer greater than 1.

At sub-step 122, an idle time of the RF pulse sequence is determined, and it is further determined whether the idle time of the RF pulse sequence is greater than a threshold. The threshold may be set based on a practical application, e.g., the threshold is equal to gating time.

At sub-step 123, when the idle time of the RF pulse sequence is no greater than the threshold, an experiential value is used to be the longitudinal magnetization intensity M_{z,pre,n-1} before the (n−1)-th characteristic tissue suppressing pulse. For example, in the case of a gating event, the experiential value is used to be the longitudinal magnetization intensity.

At sub-step 124, when the idle time of the RF pulse sequence is greater than the threshold, the longitudinal magnetization intensity M_{z,pre,n-1} before the (n−1)-th characteristic tissue suppressing pulse is calculated in real time according to the running condition of the RF pulse sequence before the (n−1)-th characteristic tissue suppressing pulse and the like. For example, when pausing time of the RF pulse sequence is long, the longitudinal magnetization intensity is calculated in real time according to the actual running condition.

In another example, an implementation way different from that in the example of FIG. 2 is provided according to a practical application. For example, the longitudinal magnetization intensity is obtained by using an experiential value or by calculating in real time based on a processing capability of a practical application system. Alternatively, the longitudinal magnetization intensity is obtained by a method determined by another condition. Step 11 can also include sub-steps not illustrated in FIG. 2.

The longitudinal magnetization intensity M_{z,pre,n-1} before the (n−1)-th characteristic tissue suppressing pulse (obtained in sub-step 124 or 123 of step 11) in sub-step 130 and the residual longitudinal magnetization intensity M_{z,res} of the characteristic tissue (in sub-step 131) may be provided for sub-step 132. At sub-step 132, inversion time TIₙ₋₁ corresponding to the (n−1)-th characteristic tissue suppressing pulse is calculated according to the longitudinal magnetization intensity M_{z,pre,n-1} and the expected residual longitudinal magnetization intensity M_{z,res} of the characteristic tissue.

At sub-step 133, the longitudinal magnetization intensity M_{z,pre,n} before the n-th characteristic tissue suppression pulse is determined. The longitudinal magnetization intensity M_{z,pre,n} is obtained according to the longitudinal magnetization intensity of the characteristic tissue suppressed by the (n−1)-th characteristic tissue suppressing pulse. The longitudinal magnetization intensity M_{z,pre,n} may be obtained by using an experiential value or by calculation in real time.

A process in sub-step 134 is similar as that in sub-step 131. In sub-step 134, an expected residual longitudinal magnetization intensity M_{z,res} provided.

A process in sub-step 135 is similar as that in sub-step 132, inversion time TIₙ corresponding to the (n)-th characteristic tissue suppressing pulse is calculated according to the longitudinal magnetization intensity M_{z,pre,n} and the expected residual longitudinal magnetization intensity M_{z,res}.

At sub-step 136, it is determined whether m inversion times are adjusted, where m is a positive integer greater than or equal to 2. For example, the longitudinal magnetization does not reach a stable state when a plurality of characteristic tissue suppressing pulses (e.g., 3 or 4) are applied at a beginning of a starting phase of the RF pulse sequence, and the respective inversion times corresponding to the characteristic tissue suppressing pulses may be adjusted. The number of the inversion times to be adjusted may be set with m, e.g., m is equal to 3 or 4. The number m of the inversion times to be adjusted may be determined according to a type of an event of disturbing the stable state in an actual RF pulse sequence, duration time and longitudinal relaxation time of a characteristic tissue.

When them inversion times are to be adjusted, it is determined in sub-step 137 whether the number of the adjusted inversion times reaches m. When the number of the adjusted inversion times reaches m at sub-step 137, the inversion time adjustment finishes, and a fixed inversion time is used to be inversion time corresponding to each of subsequent characteristic tissue suppressing pulses in sub-step 138. When the number of the adjusted inversion times does not reach m, it is determined whether the longitudinal magnetization reaches the stable state in sub-step 139.

When more than m inversion times are to be adjusted, it is determined whether the longitudinal magnetization reaches the stable state in sub-step 139. When the longitudinal magnetization reaches the stable state, the inversion time adjustment is completed, and fixed inversion time is used to be the inversion time corresponding to each of subsequent characteristic tissue suppressing pulses in sub-step 138. When the stable state is not reached, 1 is added to n in sub-step 1300. Procedures in steps 133 and 134 are returned to be performed as a cycle e.g., the inversion time corresponding to a next characteristic tissue suppressing pulse is calculated, until the inversion time adjustment is completed.

In an example, procedures in step 12 may be implemented through another method, or includes sub-steps not illustrated in figures. For example, it is determined whether the stable state is reached, but it is not determined whether m inversion times are to be adjusted and whether the number of the adjusted inversion time reaches m. Alternatively, there is no determination procedure above, and each inversion time is adjusted based on calculation.

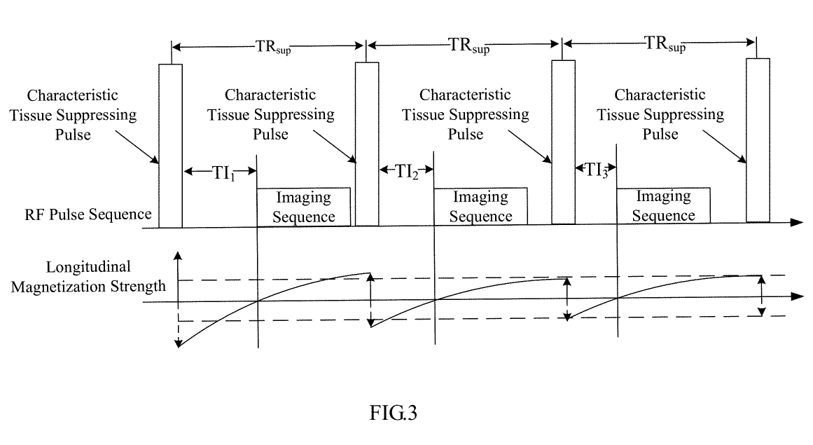

FIG. 3 is a schematic diagram illustrating an RF pulse sequence relative to time and a relaxation curve of longitudinal magnetization intensity of a characteristic tissue. In FIG. 3, respective longitudinal magnetization changing curves in three repetition time TRₛᵤₚ are different from each other, and the longitudinal magnetization is not in stable state. Three inversion times TI₁, TI₂, and TI₃ after three characteristic tissue suppressing pulses are changed when the corresponding longitudinal magnetization is changed. The three inversion times TI₁, TI₂, and TI₃ in FIG. 3 are sequentially shortened, thereby ensuring that the corresponding longitudinal magnetization intensity is equal to 0 at the end of each inversion time, and an imaging sequence may be applied.

In the example of FIG. 3, an expected residual longitudinal magnetization intensity of the characteristic tissue is 0. In another example, the expected residual longitudinal magnetization intensity of the characteristic tissue is not equal to zero. Further, four characteristic tissue suppressing pulses and three imaging sequences are illustrated in FIG. 3, and the number of the characteristic tissue suppression pulses and the number of the imaging sequences in a practical application may not be limited thereto. The characteristic tissue suppressing pulses and the imaging sequences are just illustrated by rectangular boxes in FIG. 3, and specific pulse enveloping shapes of the characteristic tissue suppression pulse and the imaging sequence are not depicted. In the practical application, the characteristic tissue suppression pulse may be a rectangular pulse, a sinc pulse, a Gaussian pulse or a Shinnar-Le Roux (SLR) pulse, and the imaging sequence may include at least one of a rectangular pulse, a sinc pulse, a windowing sinc pulse, a ramp pulse, a Gaussian pulse and an SLR pulse, which is not limited herein.

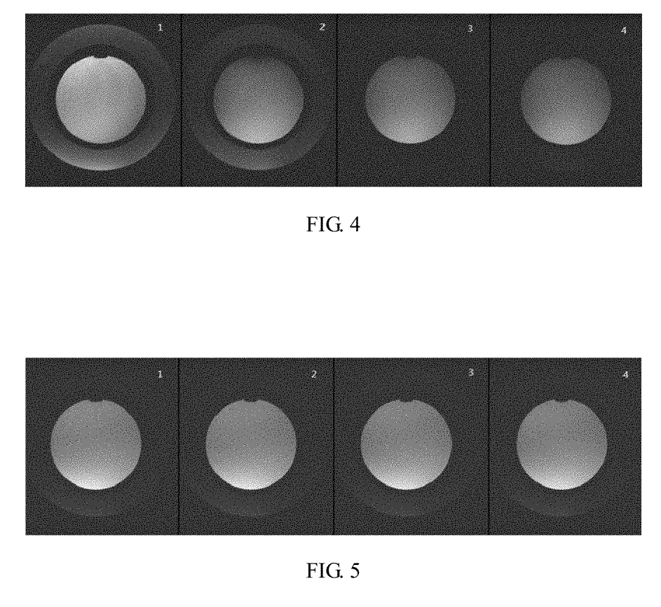

FIG. 4 is a diagram illustrating a fat signal suppressing effect by using fixed inversion time in a fat signal suppressing experiment according to an example of the present disclosure. FIG. 5 a diagram illustrating a fat signal suppressing effect by changing inversion time in a fat signal suppressing experiment according to an example of the present disclosure. The experiments of FIG. 4 and FIG. 5 were performed by using a same Spectral Adiabatic Inversion Recovery (SPAIR) pulse as the characteristic tissue suppressing pulse in the case of respiratory gating as simulated by water phantoms. The SPAIR pulse, as a frequency selective saturation pulse, is used in clinical practice because of its insensitivity to the non-uniformity of an RF exciting field.

In the experiment of FIG. 4, inversion time in the stable state is used to be the inversion time after each SPAIR pulse, e.g., fixed inversion time was used. Four layers of images of the water phantom are obtained. First to fourth layers of images are designated by 1, 2, 3, 4, respectively. In FIG. 4, the first-layer image and the second-layer image are blurry with low tissue contrasts, which have low image quality. The fat signal is not completely suppressed.

In the experiment of FIG. 5, the inversion time after a first SPAIR pulse was calculated according to the longitudinal magnetization intensity of the fat tissue before the SPAIR pulse, and the inversion times after the other three SPAIR pulses are the same as that in the stable state. A first inversion time was not equal to the subsequent three pieces of inversion time. Corresponding to four layers of images of FIG. 4, four layers of images of the water phantom are obtained in FIG. 5. First to fourth layers of images are designated by 1, 2, 3, 4, respectively. In FIG. 5, the first to fourth layers of images are all clear with high tissue contrasts and high image quality. The fat signal at each layer was completely suppressed.

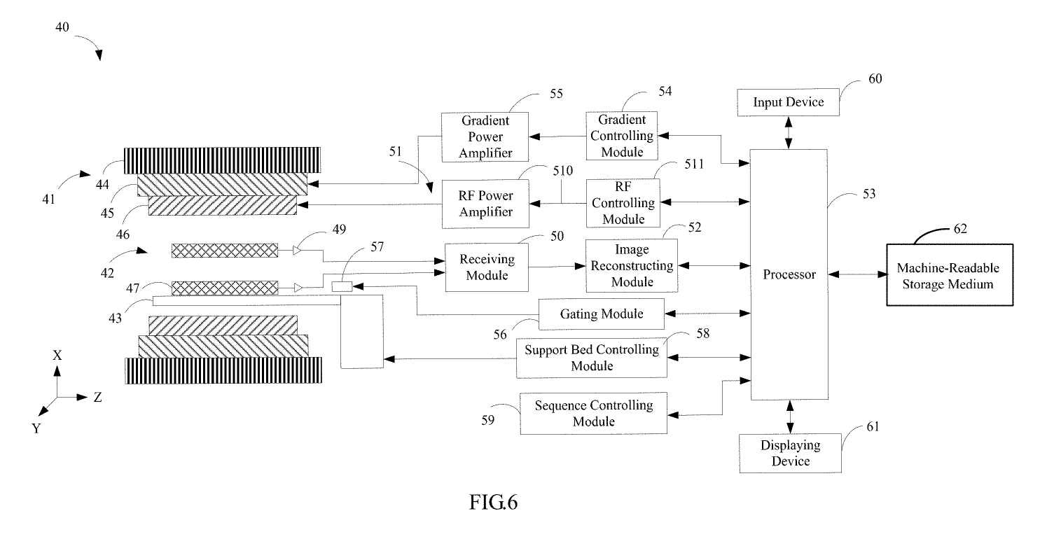

FIG. 6 illustrates a schematic diagram of a device 40 for magnetic resonance imaging according to an example of the present disclosure. The device 40 for magnetic resonance imaging includes a pulse generator 51, a processor 53, a receiving coil 47 and an image reconstructing module 52. The pulse generator 51 is configured to generate a characteristic tissue suppressing pulse, and generate an imaging sequence when inversion time elapses after the characteristic tissue suppression pulse. A processor 53 and a non-transitory machine-readable storage medium 62 storing machine executable instructions which are executable by the processor to: determine a longitudinal magnetization intensity of a characteristic tissue of a scanned subject according to a current running condition of a scanning sequence; obtain an inversion time corresponding to a characteristic tissue suppressing pulse to be generated according to the longitudinal magnetization intensity; return to determining the longitudinal magnetization intensity of the characteristic tissue of the scanned subject according to the current running condition of the scanning sequence when the scanning sequence is running. A receiving coil 47 is configured to receive an echo signal. An image reconstructing module 52 is configured to reconstruct an image according to the received echo signals when the scanning sequence finishes running. The following are detailed descriptions made in conjunction with FIG. 6.

The device 40 for magnetic resonance imaging includes a magnet assembly 41 with a cavity 42 to take in a subject lying on a support bed 43. The magnet assembly 41 includes a main magnet 44 configured to generate a static magnetic field, gradient coils 45 configured to generate gradient fields in X direction, Y direction and Z direction, and an RF transmitting coil 46 configured to transmit an RF pulse. The main magnet 44 is configured to generate a static magnetic field by using a superconducting coil. The main magnet 44 may be a permanent magnet or a resistive magnet. When the superconducting coil is used, the main magnet 44 may include a cooling system for cooling the superconducting coil, for example, a liquid helium-cooled cryostat.

The pulse generator 51 includes an RF controlling module 511, an RF power amplifier 510 and an RF transmitting coil 46. The RF controlling module 511 controls the RF transmitting coil 46 to transmit an RF pulse through the RF power amplifier 510. The RF power amplifier 510 amplifies the power of a signal output by the RF controlling module 511 and then provides the signal to the RF transmitting coil 46 so as to transmit characteristic tissue suppressing pulses and imaging sequences. The pulse generator 51 repeatedly generates the characteristic tissue suppressing pulses at an interval of repetition time to suppress a characteristic tissue signal, and for each characteristic tissue suppression pulse, generates an imaging pulse at the end of inversion time after the characteristic tissue suppression pulse. The pulse generator 51 may be connected with a sequence controlling module 59. In the example, the sequence controlling module 59 is connected with the RF controlling module 511 through the processor 53. In another example, the sequence controlling module 59 may be directly or indirectly connected with the pulse generator 51. The sequence controlling module 59 may control the RF control module 511 to control a sequence of RF pulses.

In the example, the device 40 for magnetic resonance imaging further includes a gradient controlling module 54 and a gradient power amplifier 55. The gradient controlling module 54 controls the gradient coils 45 by using the gradient power amplifier 55 to generate gradient fields. The gradient fields are overlapped on the static magnetic field to achieve spatial coding on a nuclear spin in a subject. In an example, a plurality of gradient coils 45 include three separate gradient coils for performing spatial coding in three orthogonal space directions (X direction, Y direction and Z direction). When the pulse generator 51 generates an imaging sequence, the gradient controlling module 54 controls the gradient coils 45 to generate gradient fields. The gradient controlling module 54 is connected with the sequence controlling module 59. In the example, the sequence controlling module 59 is connected with the gradient controlling module 54 through the processor 53. In another example, the sequence controlling module 59 may be directly or indirectly connected with the gradient controlling module 54. The sequence controlling module 59 may control the gradient controlling module 54 to control a gradient sequence.

The receiving coil 47 may be an array formed by receiving coil modules to receive an echo signal. The receiving coil 47 may be arranged close to the subject. An echo signal may be amplified by the amplifier 49 and the amplified echo signal is transmitted to a receiving module 50. The receiving module 50 may process and digitalize the echo signal to generate a digitalized projection intensity signal provided to the image reconstructing module 52. The image reconstructing module 52 may reconstruct an image according to the projection intensity signal.

The processor 53 is connected with a machine-readable storage medium 62. The machine-readable storage medium 62 stores machine executable instructions which are executable by the processor to: determine a longitudinal magnetization intensity of a characteristic tissue of a scanned subject according to a current running condition of a scanning sequence; determine inversion time corresponding to a characteristic tissue suppressing pulse to be generated according to the longitudinal magnetization intensity; return to determining the longitudinal magnetization intensity of the characteristic tissue of the scanned subject according to the current running condition of the scanning sequence when the scanning sequence is being run.

In an example, the processor 53 is caused by the machine-executable instructions to determine the inversion time corresponding to the characteristic tissue suppressing pulse according to the longitudinal magnetization intensity when the longitudinal magnetization of the characteristic tissue does not reaches a stable state; and set the inversion time corresponding to the characteristic tissue suppressing pulse with a fixed value when the longitudinal magnetization of the characteristic tissue reaches the stable state.

In an example, the processor 53 is caused by the machine-executable instructions to determine the inversion time according to the longitudinal magnetization intensity and an expected residual longitudinal magnetization intensity of the characteristic tissue.

In an example, the processor 53 is caused by the machine-executable instructions to when the longitudinal magnetization of the characteristic tissue does not reach a stable state, estimate the longitudinal magnetization intensity of the characteristic tissue according to a current running condition of the scanning sequence and a longitudinal magnetization intensity of the characteristic tissue in the stable state.

In an example, the longitudinal magnetization intensity of the characteristic tissue in the stable state is determined according to repetition time of the characteristic tissue suppressing pulse and longitudinal relaxation time of the characteristic tissue.

In an example, the device 40 for magnetic resonance imaging further includes a gating module 56, a gating circuit 57 and a support bed controlling module 58. The gating module 56 and the gating circuit 57 are configured to alleviate motion artifacts such as motion artifacts caused by respiration. The support bed controlling module 58 is configured to control the motion of the support bed 43.

The processor 53 is caused by the machine-executable instructions to control the gating module 56 and the support bed controlling module 58, and take charge of overall control, receive information provided by an input device 60 such as a keyboard, a mouse, a touch screen, and further convert an image reconstructed by the image reconstructing module 52 into visual image data to be displayed by a display device 61.

The receiving module 50, the image reconstructing module 52, the processor 53, the RF controlling module 511, the gradient controlling module 54, the gating module 56, the support bed controlling module 58 and the sequence controlling module 59 of the device 40 for magnetic resonance imaging may be implemented by software, or may be implemented by hardware, or may be implemented in a combination of software and hardware. The device 40 for magnetic resonance imaging may further include another element not shown in the figure, such as a memory, a power supply circuit, etc. Alternatively, in some examples, some of the elements illustrated in the figure may be omitted, for example, the gating module 56 and the gating circuit 57 may be omitted when a still part of the subject is scanned.

Since the device examples substantially correspond to the method examples, a reference may be made to part of the descriptions of the method examples for the related part. The method examples and the device examples may complement each other. The device examples described above are merely illustrative, where the units described as separate members may be or not be physically separated, and the members displayed as units may be or not be physical units, i.e., may be located in one place, or may be distributed to a plurality of network units. Part or all of the modules may be selected according to actual requirements to implement the objectives of the solutions in the present disclosure.

The terminology used in the present disclosure is for the purpose of describing a particular example only, and is not intended to limit the present disclosure. Unless otherwise defined, the technical and scientific terms used herein have the same meaning as commonly understood by one of ordinary skill in the art to which this invention belongs. The terms “a”, “an” and other words like these, as used herein and in claims, do not represent quantitative limitations and may represent presence of at least one.

Also, the term “including”, “comprising” or any variation thereof means that an element or article present before “including” or “comprising” encompass elements or articles and equivalents thereof recited behind “including” or “comprising” without excluding other elements or articles. The term “connecting”, “connected” or any variation thereof is not limited to physical or mechanical connection and may include electrical coupling, regardless of directly or indirectly. The singular forms such as “a”, ‘said”, and “the” used in the present disclosure and the appended claims are also intended to include multiple, unless the context clearly indicates otherwise. It is also to be understood that the term “and/or” as used herein refers to any or all possible combinations that include one or more associated recited items.

The foregoing disclosure is merely illustrative of preferred examples of the disclosure but not intended to limit the disclosure, and any modifications, equivalent substitutions, adaptations thereof made without departing from the spirit and scope of the disclosure shall be encompassed in the claimed scope of the appended claims.

What is claimed is:

1. A method of magnetic resonance imaging, comprising:
for each scanning step in a scanning sequence for a subject, performing operations including:
determining a longitudinal magnetization intensity of a characteristic tissue of the subject according to a current running condition of the scanning sequence;
obtaining an inversion time corresponding to a characteristic tissue suppressing pulse to be generated according to the determined longitudinal magnetization intensity;
generating the characteristic tissue suppression pulse;
generating an imaging pulse sequence when the inversion time elapses after generating the characteristic tissue suppression pulse; and
receiving an echo signal from the subject, the echo signal corresponding to the generated imaging pulse sequence; and
reconstructing an image of the subject according to the received echo signals when the scanning sequence finishes the scanning steps.

2. The method according to claim 1, wherein determining the longitudinal magnetization intensity of the characteristic tissue comprises:
in response to determining that the longitudinal magnetization of the characteristic tissue does not reach a stable state, estimating the longitudinal magnetization intensity of the characteristic tissue according to the current running condition of the scanning sequence and an expected longitudinal magnetization intensity of the characteristic tissue in the stable state.

3. The method according to claim 2, wherein the expected longitudinal magnetization intensity of the characteristic tissue in the stable state is determined according to a repetition time of the characteristic tissue suppressing pulse and a longitudinal relaxation time of the characteristic tissue.

4. The method according to claim 1, wherein determining the longitudinal magnetization intensity of the characteristic tissue comprises:
in response to determining that the longitudinal magnetization of the characteristic tissue reaches a stable state, determining the longitudinal magnetization intensity of the characteristic tissue based on a repetition time of the characteristic tissue suppressing pulse and a longitudinal relaxation time of the characteristic tissue.

5. The method according to claim 1, wherein determining the longitudinal magnetization intensity of the characteristic tissue comprises:
in response to determining that an event of disturbing a stable state of the longitudinal magnetization occurs, determining the longitudinal magnetization intensity of the characteristic tissue according to the event of disturbing the stable state of the longitudinal magnetization.

6. The method according to claim 5, wherein determining the longitudinal magnetization intensity of the characteristic tissue according to the event of disturbing the stable state of the longitudinal magnetization comprises:
determining the current running condition of the scanning sequence;
determining whether an idle time of the scanning sequence is greater than a predetermined threshold;
in response to determining that the idle time is no greater than the predetermined threshold, determining the longitudinal magnetization intensity to be an experiential value; and
in response to determining that the idle time is greater than the predetermined threshold, calculating the longitudinal magnetization intensity in real time according to the determined current running condition of the scanning sequence.

7. The method according to claim 1, wherein obtaining the inversion time corresponding to the characteristic tissue suppressing pulse to be generated according to the determined longitudinal magnetization intensity comprises:
determining the inversion time according to the determined longitudinal magnetization intensity and an expected residual longitudinal magnetization intensity of the characteristic tissue after generating the characteristic tissue suppressing pulse, such that the longitudinal magnetization intensity of the characteristic tissue is substantially equal to the expected longitudinal magnetization intensity of the characteristic tissue when the inversion time elapses after generating the characteristic tissue suppressing pulse.

8. The method according to claim 1, wherein obtaining the inversion time corresponding to the characteristic tissue suppressing pulse to be generated according to the determined longitudinal magnetization intensity comprises:
in response to determining that the longitudinal magnetization of the characteristic tissue does not reaches a stable state, determining the inversion time corresponding to the characteristic tissue suppressing pulse according to the determined longitudinal magnetization intensity; and
in response to determining that the longitudinal magnetization of the characteristic tissue reaches the stable state, setting the inversion time corresponding to the characteristic tissue suppressing pulse to be a fixed value.

9. The method according to claim 8, wherein determining the inversion time corresponding to the characteristic tissue suppressing pulse according to the determined longitudinal magnetization intensity comprises:
determining the inversion time based on the determined longitudinal magnetization intensity and an expected residual longitudinal magnetization intensity of the characteristic tissue.

10. The method according to claim 1, wherein obtaining the inversion time corresponding to the characteristic tissue suppressing pulse to be generated according to the determined longitudinal magnetization intensity comprises:
adjusting a value of the inversion time for a plurality of times;
in response to determining that a number of the plurality of times is more than a predetermined threshold, determining whether the longitudinal magnetization reaches a stable state; and
in response to determining that the longitudinal magnetization reaches the stable state, determining the inversion time to be a fixed value.

11. The method according to claim 1, wherein the scanning step is a layer when the scanning sequence is a two-dimensional scanning sequence or a slice selection direction code when the scanning sequence is a three-dimensional scanning sequence.

12. A device for magnetic resonance imaging, comprising:
a pulse generator configured to generate a characteristic tissue suppressing pulse and generate an imaging sequence when an inversion time elapses after the characteristic tissue suppression pulse;
a processor and a non-transitory machine-readable storage medium storing machine executable instructions which are executable by the processor to:
for each scanning step of a scanning sequence for a subject, determine a longitudinal magnetization intensity of a characteristic tissue of the subject according to a current running condition of the scanning sequence and obtain the inversion time corresponding to a characteristic tissue suppressing pulse to be generated according to the determined longitudinal magnetization intensity;
a receiving coil configured to receive an echo signal from the subject; and
an image reconstructing module configured to reconstruct an image according to the received echo signals when the scanning sequence finishes the scanning steps.

13. The device according to claim 12, wherein the processor is caused by the machine-executable instructions to:
in response to determining that the longitudinal magnetization of the characteristic tissue does not reach a stable state, estimate the longitudinal magnetization intensity of the characteristic tissue according to the current running condition of the scanning sequence and an expected longitudinal magnetization intensity of the characteristic tissue in the stable state.

14. The device according to claim 13, wherein the expected longitudinal magnetization intensity of the characteristic tissue in the stable state is determined according to a repetition time of the characteristic tissue suppressing pulse and a longitudinal relaxation time of the characteristic tissue.

15. The device according to claim 12, wherein the processor is caused by the machine-executable instructions to:
in response to determining that the longitudinal magnetization of the characteristic tissue reaches a stable state, determine the longitudinal magnetization intensity of the characteristic tissue based on a repetition time of the characteristic tissue suppressing pulse and a longitudinal relaxation time of the characteristic tissue.

16. The device according to claim 12, wherein the processor is caused by the machine-executable instructions to:
in response to determining that an event of disturbing a stable state of the longitudinal magnetization occurs, determine the longitudinal magnetization intensity of the characteristic tissue according to the event of disturbing the stable state of the longitudinal magnetization.

17. The device according to claim 12, wherein the processor is caused by the machine-executable instructions to:
determine the inversion time according to the determined longitudinal magnetization intensity and an expected residual longitudinal magnetization intensity of the characteristic tissue after generating the characteristic tissue suppressing pulse, such that the longitudinal magnetization intensity of the characteristic tissue is substantially equal to the expected longitudinal magnetization intensity of the characteristic tissue when the inversion time elapses after generating the characteristic tissue suppressing pulse.

18. The device according to claim 12, wherein the processor is caused by the machine-executable instructions to:
in response to determining that the longitudinal magnetization of the characteristic tissue does not reaches a stable state, determine the inversion time corresponding to the characteristic tissue suppressing pulse according to the determined longitudinal magnetization intensity; and
in response to determining that the longitudinal magnetization of the characteristic tissue reaches the stable state, set the inversion time corresponding to the characteristic tissue suppressing pulse to be a fixed value.

19. The device according to claim 18, wherein the processor is caused by the machine-executable instructions to:
determining the inversion time based on the determined longitudinal magnetization intensity and an expected residual longitudinal magnetization intensity of the characteristic tissue.

20. The device according to claim 12, wherein the processor is caused by the machine-executable instructions to:
adjust a value of the inversion time for a plurality of times;
in response to determining that a number of the plurality of times is more than a predetermined threshold, determine whether the longitudinal magnetization reaches a stable state; and
in response to determining that the longitudinal magnetization reaches the stable state, determine the inversion time to be a fixed value.
